(19) United States
(12) Patent Application Publication
(10) Pub. No.: US 2017/0325489 A1
(43) Pub. Date: Nov. 16, 2017

(54) GELATIN BASED EDIBLE ENERGY COMPOSITION

(71) Applicant: Phytonutritia, LLC, Elmwood Park, NJ (US)

(72) Inventor: Raed Omar, Franklin Lakes, NJ (US)

(21) Appl. No.: 15/156,128

(22) Filed: May 16, 2016

(51) Int. Cl. A23L 13/40; A61K 31/685; A61K 31/522; A61K 31/205; A61K 31/185; A23L 27/20; A61K 31/14; A61K 31/047; A23L 27/23; A23L 27/21; A61K 36/16; A61K 31/198

(52) U.S. Cl. CPC A23L 13/40; A61K 36/16; A61K 31/685; A61K 31/522; A61K 31/205; A61K 31/198; A23L 27/204; A61K 31/14; A61K 31/047; A23L 27/23; A23L 27/215; A61K 31/185

(57) ABSTRACT

A gelatin based edible energy composition including at least a protein based gelatin, a methylated xanthine, and a choline derivative. Other embodiments may be described and claimed including compositions with at least a flavorant and a sweetener.

### TECHNICAL FIELD

Various embodiments described herein relate to edible energy compositions.

### BACKGROUND INFORMATION

It may be desirable to provide an edible composition that improves a consumers energy and mental acuity. It may be further desirable to provide an edible composition that has non-liquid format including a semi-solid structure. It may also be desirable to include protein in the composition and control the release of elements of the composition once consumed over time. The present invention provides such compositions.

### DETAILED DESCRIPTION

An energy composition may ideally have a semi-solid food texture, enabling or requiring a User consuming (a consumer) to chew the composition prior to shallowing the composition. A semi-solid energy composition may aid diet in addition to providing energy by increasing a consumer's satiety versus a liquid based energy composition.

In an embodiment, the energy composition may include gel or gelatin in sufficient quantity to enable the energy composition to have a semi-solid consistency at room temperature. The gel or gelatin may be protein based in an embodiment and formed from various animal components including skin, bones, and connective tissues of animals including cattle, chicken, pigs, horses, fish, in an embodiment, beef bones or beef hoofs. The protein gel or gelation may be meet various cultural requirements including Kosher processing requirements or ingredients in an embodiment.

In an embodiment, the gelatin may be animal based or plant based. The animal based gelatin may include collagen including a hydrolyzed form of collagen. The plant based gelatin may be derived from the seaweed extracts including agar and carrageenan and pectin and konjac. The energy composition may also include one or more stimulants including a methylated xanthine, a choline derivative, taurine, L-Carnitine, Vitamin B3, or an nootropic ingredient including ginkgo biloba, or phosphatidylserine.

Several stimulants are derived from xanthine including caffeine and theobromine. The energy composition may one more methylated xanthines or methylxanthines including caffeine, aminophylline, IBMX, paraxanthine, pentoxifylline, theobromine, and theophylline. Caffeine may be found or derived from Coffee, Guarana, Yerba mate, Tea, Kola, Guayusa, Holly, and other natural or synthetic substances. Theobromine may be found or derived from Cacao, Yerba mate, Kola, and other natural or synthetic substances. Theophylline may be found or derived from Tea leaves, Cacao, Yerba mate, Kola, and other natural or synthetic substances.

Choline is a water-soluble nutrient where choline and its derivatives may be a nootropic or form of a “smart drug” that may increase mental acuity and affect moods due the role the neurotransmitter acetylcholine may play in various cognition systems within the brain where Choline is a chemical precursor needed to produce acetylcholine. Acetylcholine metabolism in the brain may affect memory, intelligence, and mood. Choline may be found in lecithin, which may be derived from soy or egg yolks. Choline may also be found or derived from Phosphatidylcholine, choline chloride. The energy composition may include other nootropics including Dimethylaminoethanol (DMAE), Dimethylethanolamine (DMEA), and Phosphatidylserine.

In an embodiment, the energy composition may further include taurine, L-Carnitine, L-Arginine, minerals, and various vitamins. The Vitamins may include Vitamin A (including Retinol, retinal, beta carotene), Vitamin B1 (thiamine), Vitamin B2 (Riboflavin), Vitamin B3 (Niacin), Vitamin B5 (pantothenic acid) , Vitamin B6 (pyridoxine), Vitamin B7 (Biotin), Vitamin B9 (folic acid), Vitamin B12 (Cyanocobalamin, hydroxocobalamin, methylcobalamin), Vitamin C (Ascorbic acid), Vitamin D (Cholecalciferol, Ergocalciferol), Vitamin E (Tocopherols, tocotrienols), and Vitamin K (phylloquinone, menaquinones).

The energy composition may also include water, sweetener. The sweetener may be a natural or artificial sweetener. The artificial sweeteners may include stevia, aspartame, sucralose, neotame, acesulfame potassium (Ace-K), saccharin, advantame, and cyclamates. The natural sweeteners may include glycerin and sugars including glucose, fructose, galactose, and sucrose. Glycerin also known as Glycerol and glycerine or glycerin is a simple polyol or sugar alcohol compound.

In an embodiment, the energy composition may include 1 to 4 percent of gelatin. The quantity of gelatin may vary as function of the other ingredients in the composition and may be sufficient to ensure the composition has a certain predetermined rigidity, semi-solid at room temperature. In an embodiment, the energy composition may include 15 to 45 percent of glycerin. The quantity of glycerin may vary as function of the other ingredients in the composition and may be sufficient to ensure the composition has a certain predetermined rigidity, semi-solid at room temperature or palatable taste.

In an embodiment, the energy composition may include multiple stimulants including various methylated xanthines, choline derivatives, and other nootropics. An energy composition may include 0.2 to 0.6 percent of stimulant including 0.2 to 0.6 of a methylated xanthine. An energy composition may include 0.2 to 0.6 percent of stimulant including 0.2 to 0.6 of a choline derivative. The quantity of various methylated xanthines, choline derivatives, and other nootropics may vary as function of the other ingredients in the composition and may be sufficient or limited to ensure the composition has a certain predetermined rigidity, semi-solid at room temperature or palatable taste.

In an embodiment, the energy composition may include multiple vitamins. An energy composition may include 0.2 to 0.6 percent of each included vitamin. The quantity of various vitamins may vary as function of the other ingredients in the composition and may be sufficient or limited to ensure the composition has a certain predetermined rigidity, semi-solid at room temperature or palatable taste. In an embodiment, the energy composition may include 0.5 to 4% percent of a natural or artificial sweetener. The quantity of the sweetener may vary as function of the other ingredients in the composition and may be sufficient to ensure the composition has a certain predetermined rigidity, semi-solid at room temperature or palatable taste. The energy composition may also include water. The quantity of water sufficient to ensure the composition has a certain predetermined rigidity, semi-solid at room temperature.

In an embodiment, the energy composition may include caffeine . An energy composition may include 0.2 to 0.6 percent of caffeine. The quantity of the caffeine may vary as function of the other ingredients in the composition and may be limited to ensure palatable taste. In an embodiment, the energy composition may include taurine. An energy composition may include 0.2 to 0.6 percent of taurine. The quantity of the taurine may vary as function of the other ingredients in the composition and may be limited to ensure palatable taste.

In an embodiment, the energy composition may include L-Carnitine. An energy composition may include 0.2 to 0.6 percent of L-Carnitine. The quantity of the L-Carnitine may vary as function of the other ingredients in the composition and may be limited to ensure palatable taste. In an embodiment, the energy composition may include L-arginine. An energy composition may include 0.2 to 0.6 percent of L-arginine. The quantity of the L-arginine may vary as function of the other ingredients in the composition and may be limited to ensure palatable taste.

In an embodiment, the energy composition may include Dimethylaminoethanol. An energy composition may include 0.2 to 0.6 percent of Dimethylaminoethanol. The quantity of the Dimethylaminoethanol may vary as function of the other ingredients in the composition and may be limited to ensure palatable taste. In an embodiment, the energy composition may include Ginkgo Biloba Leaf Extract. An energy composition may include 0.2 to 0.6 percent of Ginkgo Biloba Leaf Extract. The quantity of the Ginkgo Biloba Leaf Extract may vary as function of the other ingredients in the composition and may be limited to ensure palatable taste.

In an embodiment, the energy composition may include Phosphatidylserine. An energy composition may include 0.05 to 0.15 percent of Phosphatidylserine, about 0.1 percent in an embodiment. The quantity of the Phosphatidylserine may vary as function of the other ingredients in the composition and may be limited to ensure palatable taste. In an embodiment, the energy composition may include High Fructose Corn Syrup. An energy composition may include 0.5 to 4.0 percent of High Fructose Corn Syrup, about 1.0 in an embodiment. The quantity of the High Fructose Corn Syrup may vary as function of the other ingredients in the composition and may be limited to ensure palatable taste.

In an embodiment, the energy composition may include a flavorant. The flavorant may include artificial and natural flavorants and the quantity may be sufficient to make the composition palatable. In an embodiment the composition may include fruit flavorants. Such fruit flavorants include, but are not limited to artificial fruit flavorants including lemon lime flavors, orange flavors, berry flavors and natural fruit flavorants including raspberry juice concentrates, berry juice concentrates, orange concentrates, lemon juice concentrates, and other fruit concentrates or juices.

The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of, ” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In the foregoing Detailed Description, various features are grouped together in a single embodiment for the purpose of streamlining the disclosure. This method of disclosure is not to be interpreted to require more features than are expressly recited in each claim. Rather, inventive subject matter may be found in less than all features of a single disclosed embodiment.

What is claimed is:

1. An edible energy composition including:
gelatin in an amount from about 1 percent to about 4 percent of the composition;
a choline derivative in an amount from about 0.2 percent to about 0.6 percent of the composition; and
a methylated xanthine in an amount from about 0.2 percent to about 0.6 percent of the composition,
wherein the composition is semi-solid at room temperature.

2. The edible energy composition of claim 1, wherein the gelatin is essentially protein.

3. The edible energy composition of claim 1, wherein the gelatin is essentially beef protein.

4. The edible energy composition of claim 1, wherein the methylated xanthine is essentially caffeine.

5. The edible energy composition of claim 1, wherein the choline derivative is essentially dimethylaminoethanol.

6. The edible energy composition of claim 1, further including glycerin in an amount from about 15 percent to about 45 percent of the composition.

7. The edible energy composition of claim 6, further including a sweetener in an amount from about 0.5 percent to about 4 percent of the composition.

8. The edible energy composition of claim 7, further including taurine in an amount from about 0.2 percent to about 0.6 percent of the composition.

9. The edible energy composition of claim 8, further including 1-carnitine in an amount from about 0.2 percent to about 0.6 percent of the composition.

10. The edible energy composition of claim 9, further including 1-arginine in an amount from about 0.2 percent to about 0.6 percent of the composition.

11. The edible energy composition of claim 9, further including ginkgo biloba leaf extract in an amount from about 0.2 percent to about 0.6 percent of the composition.

12. The edible energy composition of claim 11, further including phosphatidylserine in an amount about 0.1 of the composition.

13. An edible energy composition consisting essentially of:
gelatin in an amount from about 1 percent to about 4 percent of the composition;
a choline derivative in an amount from about 0.2 percent to about 0.6 percent of the composition;
a methylated xanthine in an amount from about 0.2 percent to about 0.6 percent of the composition;
glycerin in an amount from about 15 percent to about 45 percent of the composition
a sweetener in an amount from about 0.5 percent to about 4 percent of the composition; and
water,
wherein the composition is semi-solid at room temperature.

14. The edible energy composition of claim 13, wherein the gelatin is essentially protein.

15. The edible energy composition of claim 13, wherein the gelatin is essentially beef protein.

16. The edible energy composition of claim 13, wherein the methylated xanthine is essentially caffeine and the choline derivative is essentially dimethylaminoethanol.

17. The edible energy composition of claim 16, further including taurine in an amount from about 0.2 percent to about 0.6 percent of the composition.

18. The edible energy composition of claim 17, further including 1-carnitine in an amount from about 0.2 percent to about 0.6 percent of the composition.

19. The edible energy composition of claim 18, further including 1-arginine in an amount from about 0.2 percent to about 0.6 percent of the composition.

20. The edible energy composition of claim 19, further including ginkgo biloba leaf extract in an amount from about 0.2 percent to about 0.6 percent of the composition and phosphatidylserine in an amount about 0.1 of the composition.
